(19) United States
(12) Patent Application Publication
(10) Pub. No.: US 2006/0286059 A1
(43) Pub. Date: Dec. 21, 2006

(54) Hair conditioning composition comprising gel matrix and high molecular weight water-soluble cationic polymer

(75) Inventors: Jian-Zhong Yang, Kobe (JP); Koji Takata, Kobe (JP)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(73) Assignee: The Procter & Gamble Company

(21) Appl. No.: 11/471,096

(22) Filed: Jun. 20, 2006

Related U.S. Application Data
(60) Provisional application No. 60/692,670, filed on Jun. 21, 2005.

(51) Int. Cl. A61K 8/81

(52) U.S. Cl. 424/70.16

(57) ABSTRACT

Disclosed is a hair conditioning composition comprising by weight: (a) from about 0.01% to about 10% of a high molecular weight water-soluble cationic polymer having a molecular weight of about 5,000,000 or more; (b) a gel matrix comprising: from about 0.1% to about 10% by weight of the composition of a cationic surfactant where in the cationic surfactant is a mono-long alkyl quaternized ammonium; from about 4.5% to about 20% by weight of the composition of a high melting point fatty compound; and an aqueous carrier. The composition of the present invention can provide another benefit such as clean rinse feel during and after rinsing while maintaining conditioning benefits of the gel matrix, and/or provide improved conditioning benefits especially wet conditioning benefits while maintaining the above dry conditioning benefits.

### CROSS REFERENCE TO RELATED APPLICATION

This application claims the benefit of U.S. Provisional Application No. 60/692,670, filed on Jun. 21, 2005.

### FIELD OF THE INVENTION

The present invention relates to a hair conditioning composition comprising: (a) from about 0.01% to about 10% of a high molecular weight water-soluble cationic polymer having a molecular weight of about 5,000,000 or more; (b) a gel matrix comprising: from about 0.1% to about 10% by weight of the composition of a cationic surfactant where in the cationic surfactant is a mono-long alkyl quaternized ammonium; from about 4.5% to about 20% by weight of the composition of a high melting point fatty compound; and an aqueous carrier. The composition of the present invention can provide another benefit such as clean rinse feel during and after rinsing while maintaining conditioning benefits of the gel matrix, and/or provide improved conditioning benefits especially wet conditioning benefits while maintaining the above dry conditioning benefits.

### BACKGROUND OF THE INVENTION

A variety of approaches have been developed to condition the hair. A common method of providing conditioning benefit is through the use of conditioning agents such as cationic surfactants and polymers, high melting point fatty compounds, low melting point oils, silicone compounds, and mixtures thereof. Most of these conditioning agents are known to provide various conditioning benefits. For example, some cationic surfactants, when used together with some high melting point fatty compounds, are believed to provide a gel matrix which is suitable for providing a variety of conditioning benefits such as slippery feel during the application to wet hair and softness and moisturized feel on dry hair.

However, there remains a need for hair conditioning compositions which provide another benefit while maintaining the above conditioning benefits of gel matrix and/or a need for hair conditioning compositions which provide improved conditioning benefits especially wet conditioning benefits while maintaining the above dry conditioning benefits.

With respect to the need for another benefit, for example, there is a need for hair conditioning compositions which provide improved clean rinse feel, while maintaining conditioning benefits of gel matrix. Hair conditioner compositions containing a gel matrix provide slippery feel during rinsing the hair, even after rinsing the hair. For consumers who prefer clean rinse feel, long-lasting slippery feels during/after rinsing the hair are not desirable, while they still prefer slippery feel during the application. One common method of obtaining clean rinse feel is rinsing the hair by a large amount of water. However, such rinsing activity provides less deposition of conditioning agents on the hair, thus less conditioning benefits to the hair. Another method of obtaining clean rinse feel is the reduction of gel matrix and/or conditioning agents. However, such reduced gel matrix and/or conditioning agents provides reduced conditioning benefits, especially reduced gel matrix provides reduced slippery feel during the application. Thus, there is a need for a hair conditioner composition which provides improved clean rinse feel during and after rinsing the hair, so that consumers can easily leave the hair and/or hands with a clean rinse feel while maintaining improved conditioning benefits of the gel matrix.

Based on the foregoing, there remains a need for hair conditioning compositions which provide another benefits while maintaining conditioning benefits of gel matrix such as slippery feel during the application to wet hair and softness and moisturized feel on dry hair, and/or a need for hair conditioning compositions which provide improved conditioning benefits especially wet conditioning benefits while maintaining the above dry conditioning benefits. Especially, there remains a need for conditioning compositions which provide improved clean rinse feel during and after rinsing the hair, while maintaining improved conditioning benefits of gel matrix such as slippery feel during the application to wet hair and softness and moisturized feel on dry hair.

None of the existing art provides all of the advantages and benefits of the present invention.

### SUMMARY OF THE INVENTION

The present invention is directed to a hair conditioning composition comprising by weight:

(a) from about 0.01% to about 10% of a high molecular weight water-soluble cationic polymer having a molecular weight of about 5,000,000 or more;

(b) a gel matrix comprising: from about 0.1% to about 10% by weight of the composition of a cationic surfactant where in the cationic surfactant is a mono-long alkyl quaternized ammonium; from about 4.5% to about 20% by weight of the composition of a high melting point fatty compound; and an aqueous carrier.

The conditioning compositions of the present invention can provide another benefit while maintaining conditioning benefits of gel matrix, and/or provide improved conditioning benefits especially wet conditioning benefits while maintaining the above dry conditioning benefits. In preferred embodiments of the present invention, the conditioning composition can provide improved clean rinse feel during and after rinsing the hair, while maintaining conditioning benefits of gel matrix such as slippery feel during the application to wet hair and softness and moisturized feel on dry hair.

These and other features, aspects, and advantages of the present invention will become better understood from a reading of the following description, and appended claims.

### DETAILED DESCRIPTION OF THE INVENTION

While the specification concludes with claims particularly pointing out and distinctly claiming the invention, it is believed that the present invention will be better understood from the following description.

Herein, “comprising” means that other steps and other ingredients which do not affect the end result can be added. This term encompasses the terms “consisting of” and “consisting essentially of”.

All percentages, parts and ratios are based upon the total weight of the compositions of the present invention, unless otherwise specified. All such weights as they pertain to listed ingredients are based on the active level and, therefore, do not include carriers or by-products that may be included in commercially available materials.

Herein, “mixtures” is meant to include a simple combination of materials and any compounds that may result from their combination.

Composition

The present invention is directed to a hair conditioning composition comprising by weight:

(a) from about 0.01% to about 10% of a high molecular weight water-soluble cationic polymer having a molecular weight of about 5,000,000 or more;

(b) a gel matrix comprising: from about 0.1% to about 10% by weight of the composition of a cationic surfactant where in the cationic surfactant is a mono-long alkyl quaternized ammonium; from about 4.5% to about 20% by weight of the composition of a high melting point fatty compound; and an aqueous carrier.

The conditioning compositions of the present invention can provide another benefit while maintaining conditioning benefits of gel matrix and/or provide improved conditioning benefits especially wet conditioning benefits while maintaining the above dry conditioning benefits. In preferred embodiments of the present invention, the conditioning composition can provide improved clean rinse feel during and after rinsing the hair, while maintaining improved conditioning benefits of gel matrix such as slippery feel during the application to wet hair and softness and moisturized feel on dry hair.

For the preferred embodiments which can provide clean feel, without intending to be limited by theory, some technical findings have been made by the inventors of the present invention as follows:

It was believed that; there is a correlation between rheological behavior (i.e., resistance to shear which is indicated by “shear stress” at a certain shear rate) of compositions and slippery feel on the wet hair, i.e., increased shear stress leads increased slippery feel, while reduced shear stress leads reduced slippery feel. It was also believed that; there is a correlation between rheological behavior of compositions and clean rinse feel, i.e., increased shear stress leads reduced clean rinse feel, while reduced shear stress leads improved clean rinse feel. Thus, it was believed that; there is also a correlation between slippery feel on the wet hair and clean rinse feel, i.e., increased slippery feel leads reduced clean rinse feel, while reduced slippery feel leads improved clean rinse feel. It has been surprisingly found that; by the combination of the high molecular weight water-soluble cationic polymer and the gel matrix comprising the mono-long alkyl quaternized ammonium salt cationic surfactant, the composition of the present invention provides reduced shear stress and improved clean rinse feel, while providing improved slippery feel during the application to wet hair. It has also been surprisingly found that; the above combination of improved clean rinse feel and improved slippery feel during the application to wet hair can not be obtained in similar gel matrix comprising other cationic surfactants such as tertiary amines, tertiary amine salts, and di-long alkyl quaternized ammonium salts. It has also been surprisingly found that; the above combination of improved clean rinse feel and improved slippery feel during the application to wet hair can not be obtained by the use of lower molecular weight polymers.

Thus, in view of providing improved clean rinse feel and improved slippery feel during the application to wet hair, it is preferred that the composition of the present invention is substantially free of other cationic surfactants than mono-long alkyl quaternized ammonium salts. Such “other cationic surfactants than mono-long alkyl quaternized ammonium salts” include tertiary amines, tertiary amine salts, and di-long alkyl quaternized ammonium salts. In the present invention, “substantially free of other cationic surfactants than mono-long alkyl quaternized ammonium salts” means that the composition contain 1% or less, preferably 0.5% or less, more preferably totally 0% of total of other cationic surfactants than mono-long alkyl quaternized ammonium salts.

Preferably, the composition of the present invention is substantially free of anionic surfactants and anionic polymers, in view of stability of the gel matrix. In the present invention, “substantially free of anionic surfactants and anionic polymers” means that the composition contains 1% or less, preferably 0.5% or less, more preferably totally 0% of total of anionic surfactants and anionic polymers.

High Molecular Weight Water-Soluble Cationic Polymer

The composition of the present invention comprises a high molecular weight water-soluble cationic polymer. The high molecular weight water-soluble cationic polymers useful herein are those having a molecular weight of about 5,000,000 or more, preferably about 10,000,000 or more, more preferably about 15,000,000 or more, in view of providing another benefit such as improved clean rinse feel during and after rinsing the hair, while maintaining conditioning benefits especially slippery feel during the application to wet hair. The molecular weight is generally up to about 50,000,000, preferably to about 40,000,000, more preferably to about 30,000,000. Many commercially available water-soluble cationic cellulose/guar polymers for the personal care use have lower molecular weight, compared to the high molecular weight water-soluble cationic polymer of the present invention. The inventors of the present invention have found that; the addition of lower molecular weight polymer may provide clean rinse feel, however, it also provide reduced wet conditioning benefits, especially reduced slippery feel during the application to wet hair.

In view of providing another benefit such as improved clean rinse feel during and after rinsing the hair, while maintaining conditioning benefits especially slippery feel during the application to wet hair, it is preferred that the high molecular weight water-soluble cationic polymers have a molar percentage of the cationic monomer unit of preferably from about 20% to about 100%, more preferably from about 30% to about 100%, still more preferably from about 50% to about 100%. Many commercially available water-soluble cationic cellulose/guar polymers for the personal care use have lower molar percentage of cationic monomer unit, compared to the high molecular weight water-soluble cationic polymer of the present invention.

In the high molecular weight water-soluble cationic polymers useful herein, preferred are non-crosslinked polymers compared to crosslinked polymers such as polyquaternium-37 in view of providing clean feel.

The polymers useful herein are water-soluble. In the present invention, “high molecular weight water-soluble cationic polymers” means that the polymer contains monomer units having positively charged functional groups and has a solubility of greater than 0.1 g/100 g water, preferably 0.5 g/100 g water at 25° C.

The high molecular weight water-soluble cationic polymers useful herein comprise a cationic water-soluble monomer. The high molecular weight water-soluble cationic polymers useful herein can further comprise a nonionic water-soluble monomer. In the present invention, “water-soluble monomers” means that the monomers have hydrophilic functional groups. Preferred hydrophilic functional groups include, for example, quaternary amine, tertiary amine, and amide, and more preferred are quaternary amine and amide.

The high molecular weight water-soluble cationic polymers useful herein are those being substantially free of anionic monomers, in view of compatibility with the cationic surfactant-containing gel matrix. In the present invention, “substantially free of anionic monomers” means that the high molecular weight water-soluble cationic polymer contains 1% or less, preferably 0.5% or less, more preferably totally 0% of total of anionic monomers.

The cationic water-soluble monomers useful herein include, for example, cationic acryl vinyl monomers, cationic vinyl benzyl monomers, and cationic dialkyldiallylvinyl monomers. The cationic acryl vinyl monomers useful herein include, for example, (meth)acrylates containing a quaternary nitrogen such as 2-(meth)acryloyloxyethyltrimethylammonium chloride and 2-(meth)acryloyloxyethyltrimethylammonium methosulfate; salts of (meth)acrylates containing a tertiary nitrogen such as 2-dimethylaminoethyl(meth)acrylate phosphate and 2-diethylaminoethyl(meth)acrylate hydrochloride; (meth)acrylamides containing a quaternary nitrogen such as 2-(meth)acryloylaminoethyltrimethylammonium methosulfate; and salts of (meth)acrylamides containing a tertiary nitrogen such as dimethylaminoethyl(meth)acrylamide hydrochloride. The cationic vinyl benzyl monomers useful herein include, for example, vinylbenzyltrialkylammonium salts such as vinylbenzyltrimethylammonium chloride. The cationic dialkyldiallylvinyl monomers useful herein include, for example, dialkyldiallylammonium salts such as dimethyldiallylammonium chloride. Among the above cationic water-soluble monomers, preferred is 2-(meth)acryloyloxyethyltrimethylammonium chloride.

The nonionic water-soluble monomers useful herein include, for example, nonionic vinyl monomers containing a carbamoyl group such as (meth)acrylamide and N,N-dimethyl(meth)acrylamide; nonionic vinyl monomers containing a hydroxyl group such as hydroxyalkyl(meth)acrylate including, but not limited to, hydroxyethyl(meth)acrylate; and other nonionic vinyl monomers such as vinylpyrrolidone. Among the above nonionic water-soluble monomers, preferred is (meth)acrylamide.

In the present invention, highly preferred high molecular weight water-soluble cationic polymer is poly(2-acryloyloxyethyltrimethyl ammonium chloride-co-acrylamide) which is a non-crosslinked polymer and wherein 2-acryloyloxyethyltrimethyl ammonium chloride is a cationic water-soluble monomer and acrylamide is a nonionic water-soluble monomer. Such polymer can be obtained from, for example, Sanyo Chemical in Japan and Ondeo Nalco.

The high molecular weight water-soluble cationic polymers are included in the composition at a level by weight of from about 0.01% to about 10%, preferably from about 0.02% to about 5%, more preferably from about 0.03% to about 0.5%, in view of providing another benefit such as improved clean rinse feel during and after rinsing the hair, while maintaining conditioning benefits especially slippery feel during the application to wet hair.

Gel Matrix

The composition of the present invention comprises a gel matrix. The gel matrix comprises a cationic surfactant, a high melting point fatty compound, and an aqueous carrier.

The gel matrix is suitable for providing various conditioning benefits such as slippery feel during the application to wet hair and softness and moisturized feel on dry hair. In view of providing the above gel matrix, the cationic surfactant and the high melting point fatty compound are contained at a level such that the weight ratio of the cationic surfactant to the high melting point fatty compound is in the range of, preferably from about 1:1 to about 1:10, more preferably from about 1:1 to about 1:6.

For forming gel matrix, it is preferred to prepare the composition by the following method:

Water is typically heated to at least about 70° C., preferably between about 80° C. and about 90° C. The cationic surfactant and the high melting point fatty compound are combined with the water to form a mixture. The temperature of the mixture is preferably maintained at a temperature higher than both the melting temperature of the cationic surfactant and the melting temperature of the high melting point fatty compound, and the entire mixture is homogenized. After mixing until no solids are observed, the mixture is gradually cooled (e.g., at a rate of from about 1° C./minute to about 5° C./minute) to a temperature below 60° C., preferably less than about 55° C. During this gradual cooling process, a significant viscosity increase is observed at between about 55° C. and about 75° C. This indicates the formation of gel matrix. The high molecular weight water-soluble cationic polymer can be added to the mixture with agitation at about 55° C., or prior to the cooling down. Additional components are then combined with the gel matrix, and cooled to room temperature.

Preferably, the present invention comprises, by weight of the hair care composition, from about 60% to about 99%, preferably from about 70% to about 95%, and more preferably from about 80% to about 95% of a gel matrix, to which optional ingredients such as silicones can be added. The composition containing the above amount of gel matrix is typically characterized by a viscosity of from about 5,000 cps to about 40,000 cps, preferably from about 10,000 cps to about 30,000 cps, and more preferably from about 12,000 cps to about 28,000 cps, as measured at 25° C., by means of a Brookfield Viscometer at shear rate of 1.0 rpm. Although the composition of the present invention can contain a thickening polymer, the composition of the present invention can have the above viscosity without the presence of any thickening polymer.

Cationic Surfactant

The cationic surfactant useful herein is a mono-long alkyl quaternized ammonium salt having the formula (I): 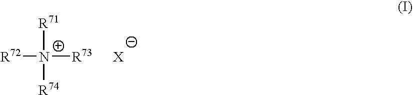
wherein one of R⁷¹, R⁷², R⁷³ and R⁷⁴ is selected from an aliphatic group of from 12 to 30 carbon atoms or an aromatic, alkoxy, polyoxyalkylene, alkylamido, hydroxyalkyl, aryl or alkylaryl group having up to about 22 carbon atoms; the remainder of R⁷¹, R⁷², R⁷³ and R⁷⁴ are independently selected from an aliphatic group of from 1 to about 8 carbon atoms or an aromatic, alkoxy, polyoxyalkylene, alkylamido, hydroxyalkyl, aryl or alkylaryl group having up to about 8 carbon atoms; and X⁻ is a salt-forming anion such as those selected from halogen, (e.g. chloride, bromide), acetate, citrate, lactate, glycolate, phosphate, nitrate, sulfonate, sulfate, alkylsulfate, and alkyl sulfonate radicals. The aliphatic groups can contain, in addition to carbon and hydrogen atoms, ether linkages, and other groups such as amino groups. The longer chain aliphatic groups, e.g., those of about 12 carbons, or higher, can be saturated or unsaturated. Preferably, one of R⁷¹, R⁷², R⁷³ and R⁷⁴ is selected from an alkyl group of from 12 to 30 carbon atoms, more preferably from 16 to 22 carbon atoms, still more preferably from 18 to 22 carbon atoms, even more preferably 22 carbon atoms; the remainder of R⁷¹, R⁷², R⁷³ and R⁷⁴ are independently selected from CH₃, C₂H₅, C₂H₄OH, CH₂C₆H₅, and mixtures thereof; and X is selected from the group consisting of Cl, Br, CH₃OSO₃, C₂H₅OSO₃, and mixtures thereof.

Nonlimiting examples of such mono-long alkyl quaternized ammonium salt cationic surfactants include: behenyl trimethyl ammonium chloride; stearyl trimethyl ammonium chloride; cetyl trimethyl ammonium chloride; hydrogenated tallow alkyl trimethyl ammonium chloride; stearyl dimethyl benzyl ammonium chloride; and stearoyl amidopropyl dimethyl benzyl ammonium chloride.

Among them, more preferred cationic surfactants are those having a longer alkyl group, i.e., C18-22 alkyl group. Such cationic surfactants include, for example, behenyl trimethyl ammonium chloride and stearyl trimethyl ammonium chloride, and still more preferred is behenyl trimethyl ammonium chloride. It is believed that; cationic surfactants having a longer alkyl group provide improved deposition on the hair, thus can provide improved conditioning benefits such as improved softness on dry hair, compared to cationic surfactant having a shorter alkyl group. It is also believed that such cationic surfactants can provide reduced irritation, compared to cationic surfactants having a shorter alkyl group.

The cationic surfactant is included in the composition at a level by weight of from about 0.1% to about 10%, preferably from about 1% to about 8%, more preferably from about 2% to about 5%, in view of providing improved conditioning benefits such as slippery feel during the application to wet hair, softness and moisturized feel on dry hair.

High Melting Point Fatty Compound

The high melting point fatty compound useful herein have a melting point of 25° C. or higher, and is selected from the group consisting of fatty alcohols, fatty acids, fatty alcohol derivatives, fatty acid derivatives, and mixtures thereof. It is understood by the artisan that the compounds disclosed in this section of the specification can in some instances fall into more than one classification, e.g., some fatty alcohol derivatives can also be classified as fatty acid derivatives. However, a given classification is not intended to be a limitation on that particular compound, but is done so for convenience of classification and nomenclature. Further, it is understood by the artisan that, depending on the number and position of double bonds, and length and position of the branches, certain compounds having certain required carbon atoms may have a melting point of less than 25° C. Such compounds of low melting point are not intended to be included in this section. Nonlimiting examples of the high melting point compounds are found in International Cosmetic Ingredient Dictionary, Fifth Edition, 1993, and CTFA Cosmetic Ingredient Handbook, Second Edition, 1992.

Among a variety of high melting point fatty compounds, fatty alcohols are preferably used in the composition of the present invention. The fatty alcohols useful herein are those having from about 14 to about 30 carbon atoms, preferably from about 16 to about 22 carbon atoms. These fatty alcohols are saturated and can be straight or branched chain alcohols. Preferred fatty alcohols include, for example, cetyl alcohol, stearyl alcohol, behenyl alcohol, and mixtures thereof.

High melting point fatty compounds of a single compound of high purity are preferred. Single compounds of pure fatty alcohols selected from the group of pure cetyl alcohol, stearyl alcohol, and behenyl alcohol are highly preferred. By “pure” herein, what is meant is that the compound has a purity of at least about 90%, preferably at least about 95%. These single compounds of high purity provide good rinsability from the hair when the consumer rinses off the composition.

The high melting point fatty compound is included in the composition at a level of from about 4.5% to about 20%, preferably from about 5.5% to about 15%, more preferably from about 6% to about 8% by weight of the composition, in view of providing improved conditioning benefits such as slippery feel during the application to wet hair, softness and moisturized feel on dry hair.

Aqueous Carrier

The conditioning composition of the present invention comprises an aqueous carrier. The level and species of the carrier are selected according to the compatibility with other components, and other desired characteristic of the product.

The carrier useful in the present invention includes water and water solutions of lower alkyl alcohols and polyhydric alcohols. The lower alkyl alcohols useful herein are monohydric alcohols having 1 to 6 carbons, more preferably ethanol and isopropanol. The polyhydric alcohols useful herein include propylene glycol, hexylene glycol, glycerin, and propane diol.

Preferably, the aqueous carrier is substantially water. Deionized water is preferably used. Water from natural sources including mineral cations can also be used, depending on the desired characteristic of the product. Generally, the compositions of the present invention comprise from about 20% to about 99%, preferably from about 30% to about 95%, and more preferably from about 80% to about 95% water.

Silicone Compound

Preferably, the compositions of the present invention preferably contain a silicone compound. It is believed that the silicone compound can provide smoothness and softness on dry hair. The silicone compounds herein can be used at levels by weight of the composition of preferably from about 0.1% to about 20%, more preferably from about 0.5% to about 10%, still more preferably from about 1% to about 8%.

The silicone compounds useful herein, as a single compound, as a blend or mixture of at least two silicone compounds, or as a blend or mixture of at least one silicone compound and at least one solvent, have a viscosity of preferably from about 1,000 to about 2,000,000 mPa·s at 25° C.

The viscosity can be measured by means of a glass capillary viscometer as set forth in Dow Corning Corporate Test Method CTM0004, Jul. 20, 1970. Suitable silicone fluids include polyalkyl siloxanes, polyaryl siloxanes, polyalkylaryl siloxanes, polyether siloxane copolymers, amino substituted silicones, quaternized silicones, and mixtures thereof. Other nonvolatile silicone compounds having conditioning properties can also be used.

Preferably, the silicone compounds have an average particle size of from about 1 microns to about 50 microns, in the composition.

The silicone compounds useful herein include polyalkyl or polyaryl siloxanes with the following structure: 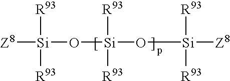
wherein R⁹³ is alkyl or aryl, and p is an integer from about 7 to about 8,000. Z⁸ represents groups which block the ends of the silicone chains. The alkyl or aryl groups substituted on the siloxane chain (R⁹³) or at the ends of the siloxane chains Z⁸ can have any structure as long as the resulting silicone remains fluid at room temperature, is dispersible, is neither irritating, toxic nor otherwise harmful when applied to the hair, is compatible with the other components of the composition, is chemically stable under normal use and storage conditions, and is capable of being deposited on and conditions the hair. Suitable Z⁸ groups include hydroxy, methyl, methoxy, ethoxy, propoxy, and aryloxy. The two R⁹³ groups on the silicon atom may represent the same group or different groups. Preferably, the two R⁹³ groups represent the same group. Suitable R⁹³ groups include methyl, ethyl, propyl, phenyl, methylphenyl and phenylmethyl. The preferred silicone compounds are polydimethylsiloxane, polydiethylsiloxane, and polymethylphenylsiloxane. Polydimethylsiloxane, which is also known as dimethicone, is especially preferred. The polyalkylsiloxanes that can be used include, for example, polydimethylsiloxanes. These silicone compounds are available, for example, from the General Electric Company in their Viscasil® and TSF 451 series, and from Dow Corning in their Dow Corning SH200 series.

The above polyalkylsiloxanes are available, for example, as a mixture with silicone compounds having a lower viscosity. Such mixtures have a viscosity of preferably from about 1,000 mPa·s to about 100,000 mPa·s, more preferably from about 5,000 mPa·s to about 50,000 mPa·s. Such mixtures preferably comprise: (i) a first silicone having a viscosity of from about 1000,000 mPa·s to about 30,000,000 mPa·s at 25° C., preferably from about 100,000 m Pa·s to about 20,000,000 mPa·s; and (ii) a second silicone having a viscosity of from about 5 mPa·s to about 10,000 mPa·s at 25° C., preferably from about 5 mPa·s to about 5,000 mPa·s. Such mixtures useful herein include, for example, a blend of dimethicone having a viscosity of 18,000,000 mPa·s and dimethicone having a viscosity of 200 mPa·s available from GE Toshiba, and a blend of dimethicone having a viscosity of 18,000,000 mPa·s and cyclopentasiloxane available from GE Toshiba.

The silicone compounds useful herein also include a silicone gum. The term “silicone gum”, as used herein, means a polyorganosiloxane material having a viscosity at 25° C. of greater than or equal to 1,000,000 centistokes. It is recognized that the silicone gums described herein can also have some overlap with the above-disclosed silicone compounds. This overlap is not intended as a limitation on any of these materials. The “silicone gums” will typically have a mass molecular weight in excess of about 200,000, generally between about 200,000 and about 1,000,000. Specific examples include polydimethylsiloxane, poly(dimethylsiloxane methylvinylsiloxane) copolymer, poly(dimethylsiloxane diphenylsiloxane methylvinylsiloxane) copolymer and mixtures thereof. The silicone gums are available, for example, as a mixture with silicone compounds having a lower viscosity. Such mixtures useful herein include, for example, Gum/Cyclomethicone blend available from Shin-Etsu.

The silicone compounds that can be used include, for example, a polypropylene oxide modified polydimethylsiloxane although ethylene oxide or mixtures of ethylene oxide and propylene oxide can also be used. The ethylene oxide and polypropylene oxide level should be sufficiently low so as not to interfere with the dispersibility characteristics of the silicone. These materials are also known as dimethicone copolyols.

Silicone compounds useful herein also include amino substituted materials. Preferred aminosilicones include, for example, those which conform to the general formula (I):
(R₁)ₐG₃₋ₐ—Si—(—OSiG₂)ₙ—(—OSiG_{b}(R₁)_{2-b})ₘ—O—SiG₃₋ₐ(R₁)ₐ
wherein G is hydrogen, phenyl, hydroxy, or C₁-C₈ alkyl, preferably methyl; a is 0 or an integer having a value from 1 to 3, preferably 1; b is 0, 1 or 2, preferably 1; n is a number from 0 to 1,999; m is an integer from 0 to 1,999; the sum of n and m is a number from 1 to 2,000; a and m are not both 0; R₁ is a monovalent radical conforming to the general formula CqH_{2q}L, wherein q is an integer having a value from 2 to 8 and L is selected from the following groups: —N(R₂)CH₂—CH₂—N(R₂)₂; —N(R₂)₂; —N(R₂)₃A⁻; —N(R₂)CH₂—CH₂—NR₂H₂A⁻; wherein R₂ is hydrogen, phenyl, benzyl, or a saturated hydrocarbon radical, preferably an alkyl radical from about C₁ to about C₂₀; A⁻ is a halide ion.

One highly preferred amino silicones are those corresponding to formula (III) wherein m=0, a=1, q=3, G=methyl, n is preferably from about 1500 to about 1700, more preferably 1600; and L is —N(CH₃)₂ or —NH₂, more preferably —NH₂. Another highly preferred amino silicones are those corresponding to formula (III) wherein m=0, a=1, q=3, G=methyl, n is preferably from about 400 to about 600, more preferably about 500; and L is —N(CH₃)₂ or —NH₂, more preferably —NH₂. Such highly preferred amino silicones can be called as terminal aminosilicones; as one or both ends of the silicone chain are terminated by nitrogen containing group.

The above aminosilicones, when incorporated into the composition, can be mixed with solvent having a lower viscosity. Such solvents include, for example, polar or non-polar, volatile or non-volatile oils. Such oils include, for example, silicone oils, hydrocarbons, and esters. Among such a variety of solvents, preferred are those selected from the group consisting of non-polar, volatile hydrocarbons, volatile cyclic silicones, non-volatile linear silicones, and mixtures thereof. The non-volatile linear silicones useful herein are those having a viscosity of from about 1 to about 20,000 centistokes, preferably from about 20 to about 10,000 centistokes at 25° C. Among the preferred solvents, highly preferred are non-polar, volatile hydrocarbons, especially non-polar, volatile isoparaffins, in view of reducing the viscosity of the aminosilicones and providing improved hair conditioning benefits such as reduced friction on dry hair. Such mixtures have a viscosity of preferably from about 1,000 mPa·s to about 100,000 mPa·s, more preferably from about 5,000 mPa·s to about 50,000 mPa·s.

Other suitable alkylamino substituted silicone compounds include those represented by the following structure: 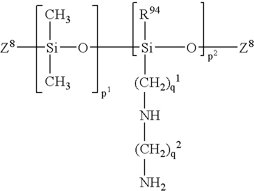
wherein R⁹⁴ is H, CH₃ or OH; p¹ and p² are integers of 1 or above, and wherein sum of p¹ and p² is from 650 to 1,500; q¹ and q² are integers of from 1 to 10. Z⁸ represents groups which block the ends of the silicone chains. Suitable Z⁸ groups include hydroxy, methyl, methoxy, ethoxy, propoxy, and aryloxy. Highly preferred are those known as “amodimethicone”. Commercially available amodimethicones useful herein include, for example, BY16-872 available from Dow Corning.

Other amino substituted silicone polymers which can be used are represented by the formula:

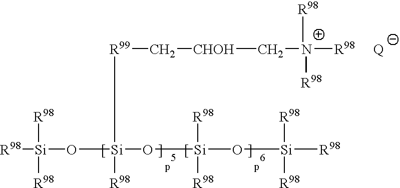

where R⁹⁸ denotes a monovalent hydrocarbon radical having from 1 to 18 carbon atoms, preferably an alkyl or alkenyl radical such as methyl; R⁹⁹ denotes a hydrocarbon radical, preferably a C₁-C₁₈ alkylene radical or a C₁-C₁₈, and more preferably C₁-C₈, alkyleneoxy radical; Q⁻ is a halide ion, preferably chloride; p⁵ denotes an average statistical value from 2 to 20, preferably from 2 to 8; p⁶ denotes an average statistical value from 20 to 200, and preferably from 20 to 50.

The silicone compounds may further be incorporated in the present composition in the form of an emulsion, wherein the emulsion is made my mechanical mixing, or in the stage of synthesis through emulsion polymerization, with or without the aid of a surfactant selected from anionic surfactants, nonionic surfactants, cationic surfactants, and mixtures thereof.

Additional Components

The composition of the present invention may include other additional components, which may be selected by the artisan according to the desired characteristics of the final product and which are suitable for rendering the composition more cosmetically or aesthetically acceptable or to provide them with additional usage benefits. Such other additional components generally are used individually at levels of from about 0.001% to about 10%, preferably up to about 5% by weight of the composition.

A wide variety of other additional components can be formulated into the present compositions. These include: other conditioning agents such as hydrolysed collagen with tradename Peptein 2000 available from Hormel, vitamin E with tradename Emix-d available from Eisai, panthenol available from Roche, panthenyl ethyl ether available from Roche, hydrolysed keratin, proteins, plant extracts, and nutrients; preservatives such as benzyl alcohol, methyl paraben, propyl paraben and imidazolidinyl urea; pH adjusting agents, such as citric acid, sodium citrate, succinic acid, phosphoric acid, sodium hydroxide, sodium carbonate; salts, in general, such as potassium acetate and sodium chloride; coloring agents, such as any of the FD&C or D&C dyes; perfumes; and sequestering agents, such as disodium ethylenediamine tetra-acetate; ultraviolet and infrared screening and absorbing agents such as octyl salicylate; and antidandruff agents such as zinc pyrithione and salicylic acid.

Low Melting Point Oil

Low melting point oils useful herein are those having a melting point of less than 25° C. The low melting point oil useful herein is selected from the group consisting of: hydrocarbon having from 10 to about 40 carbon atoms; unsaturated fatty alcohols having from about 10 to about 30 carbon atoms such as oleyl alcohol; unsaturated fatty acids having from about 10 to about 30 carbon atoms; fatty acid derivatives; fatty alcohol derivatives; ester oils such as pentaerythritol ester oils, trimethylol ester oils, citrate ester oils, and glyceryl ester oils; poly α-olefin oils; and mixtures thereof. Preferred low melting point oils herein are selected from the group consisting of: ester oils such as pentaerythritol ester oils, trimethylol ester oils, citrate ester oils, and glyceryl ester oils; poly α-olefin oils; and mixtures thereof, Particularly useful pentaerythritol ester oils and trimethylol ester oils herein include pentaerythritol tetraisostearate, pentaerythritol tetraoleate, trimethylolpropane triisostearate, trimethylolpropane trioleate, and mixtures thereof. Particularly useful citrate ester oils herein include triisocetyl citrate, triisostearyl citrate, and trioctyldodecyl citrate. Particularly useful glyceryl ester oils herein include triisostearin, triolein, and trilinolein. Particularly useful poly α-olefin oils herein include polydecenes with tradenames PURESYN 6 having a number average molecular weight of about 500 and PURESYN 100 having a number average molecular weight of about 3000 and PURESYN 300 having a number average molecular weight of about 6000 available from Exxon Mobil Co.

Cationic Conditioning Polymer

Cationic conditioning polymers useful herein are those having an average molecular weight of at least about 5,000, typically from about 10,000, preferably from about 100,000 to about 2 million. Cationic conditioning polymers useful herein are not polymers described above under the tile “HIGH MOLECULAR WEIGHT WATER-SOLUBLE CATIONIC POLYMER”.

Suitable cationic polymers include, for example, copolymers of vinyl monomers having cationic amine or quaternary ammonium functionalities with water soluble spacer monomers such as acrylamide, methacrylamide, alkyl and dialkyl acrylamides, alkyl and dialkyl methacrylamides, alkyl acrylate, alkyl methacrylate, vinyl caprolactone, and vinyl pyrrolidone. Other suitable spacer monomers include vinyl esters, vinyl alcohol (made by hydrolysis of polyvinyl acetate), maleic anhydride, propylene glycol, and ethylene glycol. Other suitable cationic polymers useful herein include, for example, cationic celluloses, cationic starches, and cationic guar gums.

Polyethylene Glycol

Polyethylene glycol can also be used as an additional component. The polyethylene glycols useful herein that are especially preferred are PEG-2M wherein n has an average value of about 2,000 (PEG-2M is also known as PEG-2,000); PEG-5M wherein n has an average value of about 5,000 (PEG-5M is also known as PEG-5,000 and Polyethylene Glycol 300,000); PEG-7M wherein n has an average value of about 7,000; PEG-9M wherein n has an average value of about 9,000; and PEG-14M wherein n has an average value of about 14,000.

Product Forms

The conditioning compositions of the present invention can be in the form of rinse-off products or leave-on products, and can be formulated in a wide variety of product forms, including but not limited to creams, gels, emulsions, mousses and sprays.

The conditioning composition of the present invention is especially suitable for rinse-off hair conditioner. Such compositions are preferably used by following steps:

(i) after shampooing hair, applying to the hair an effective amount of the conditioning compositions for conditioning the hair; and

(ii) then rinsing the hair.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention. Where applicable, ingredients are identified by chemical or CTFA name, or otherwise defined below.

[Compositions]

| Components | Ex. 1 | Ex. 2 | Ex. 3 | Ex. 4 | Ex. 5 | Ex. 6 | Ex. 7 | Ex. 8 |
|---|---|---|---|---|---|---|---|---|
| Behenyl trimethyl ammonium | 2.25 | 2.25 | — | — | 3.38 | 1.80 | 2.25 | 2.25 |
| chloride | | | | | | | | |
| Behenyl trimethyl ammonium | — | — | — | 3.45 | — | — | — | — |
| methosulfate | | | | | | | | |
| Stearyl trimethyl ammonium | — | — | 2.00 | — | — | — | — | — |
| chloride | | | | | | | | |
| Cetyl alcohol | 1.88 | 1.86 | 1.65 | 1.97 | 2.23 | 1.49 | 1.88 | 1.88 |
| Stearyl alcohol | 4.64 | 4.64 | 4.08 | 3.55 | 4.18 | 3.71 | 4.64 | 3.00 |
| Behenyl alcohol | — | — | — | — | — | — | — | 0.50 |
| High molecular weight water- | 0.15 | 0.05 | 0.05 | — | 0.03 | 0.50 | 0.02 | 0.35 |
| soluble cationic polymer-1 *1 | | | | | | | | |
| High molecular weight water- | — | — | — | 0.05 | — | — | — | — |
| soluble cationic polymer-6 *6 | | | | | | | | |
| Dimethicone blend *7 | — | — | — | — | — | 4.20 | — | — |
| Dimethicone/Cyclomethicone *8 | — | — | — | — | — | — | 4.20 | — |
| Aminosilicone-1 *9 | 3.50 | 3.50 | 3.50 | 3.00 | 4.00 | — | — | 2.00 |
| Isopropanol | 0.28 | 0.22 | 0.25 | 0.34 | — | — | — | 0.50 |
| Disodium EDTA | 0.13 | 0.13 | 0.13 | 0.13 | 0.13 | 0.13 | 0.13 | 0.13 |
| Benzyl alcohol | 0.4 | 0.4 | 0.4 | 0.4 | 0.4 | 0.4 | 0.4 | 0.4 |
| Kathon CG *10 | 0.033 | 0.033 | 0.033 | 0.033 | 0.033 | 0.033 | 0.033 | 0.033 |
| Perfume | 0.35 | 0.35 | 0.25 | 0.25 | 0.25 | 0.25 | 0.25 | 0.25 |
| Panthenol | 0.05 | 0.05 | 0.05 | — | — | — | 0.05 | 0.05 |
| Panthenyl ethyl ether | 0.05 | 0.05 | 0.05 | — | — | — | 0.05 | 0.05 |
| Vitamin E | — | — | — | — | 0.01 | 0.01 | 0.01 | 0.01 |
| Octyl methoxycinnamate | — | — | — | — | — | — | 0.09 | 0.09 |
| Benzophenone-3 | — | — | — | — | — | — | 0.09 | 0.09 |

| | |
|---|---|
| Deionized Water | q.s. to 100% |

| Components | Ex. 9 | Ex. 10 | Ex. 11 | Ex. 12 | Ex. 13 | Ex. 14 | Ex. 15 | Ex. 16 | Ex. 17 |
|---|---|---|---|---|---|---|---|---|---|
| Behenyl trimethyl ammonium | 2.25 | 2.25 | 3.45 | 3.38 | 1.80 | 2.25 | 2.25 | 2.25 | |
| chloride | | | | | | | | | |
| Behenyl trimethyl ammonium | | | | | | | | | 1.58 |
| methosulfate | | | | | | | | | |
| Cetyl alcohol | 1.88 | 1.86 | 1.97 | 2.23 | 1.49 | 1.88 | 1.88 | 1.86 | 1.30 |
| Stearyl alcohol | 4.64 | 4.64 | 3.55 | 4.18 | 3.71 | 4.64 | 4.64 | 4.64 | 3.25 |
| High molecular weight water- | 0.15 | 0.05 | — | — | — | — | — | — | — |
| soluble cationic polymer-2 *2 | | | | | | | | | |
| High molecular weight water- | — | — | 0.1 | 0.05 | — | — | — | — | — |
| soluble cationic polymer-3 *3 | | | | | | | | | |
| High molecular weight water- | — | — | — | — | 0.2 | 0.07 | — | — | — |
| soluble cationic polymer-4 *4 | | | | | | | | | |
| High molecular weight water- | — | — | — | — | — | — | 0.12 | 0.03 | 0.08 |
| soluble cationic polymer-5 *5 | | | | | | | | | |
| Dimethicone blend *7 | — | — | — | — | — | 4.20 | — | — | — |
| Dimethicone/Cyclomethicone *8 | — | — | — | — | — | — | 4.20 | — | — |
| Aminosilicone-1 *9 | 3.50 | 3.50 | 3.50 | 3.00 | 4.00 | — | — | 2.00 | — |
| Aminosilicone-2 *11 | — | — | — | — | — | — | — | — | 1.5 |
| Isopropanol | 0.28 | 0.22 | 0.25 | 0.34 | — | — | — | 0.50 | 0.20 |
| Disodium EDTA | 0.13 | 0.13 | 0.13 | 0.13 | 0.13 | 0.13 | 0.13 | 0.13 | 0.13 |
| Benzyl alcohol | 0.4 | 0.4 | 0.4 | 0.4 | 0.4 | 0.4 | 0.4 | 0.4 | 0.4 |
| Kathon CG *10 | 0.033 | 0.033 | 0.033 | 0.033 | 0.033 | 0.033 | 0.033 | 0.033 | 0.033 |
| Perfume | 0.35 | 0.35 | 0.25 | 0.25 | 0.25 | 0.25 | 0.25 | 0.25 | 0.35 |
| Panthenol | 0.05 | 0.05 | 0.05 | — | — | — | 0.05 | 0.05 | 0.05 |
| Panthenyl ethyl ether | 0.05 | 0.05 | 0.05 | — | — | — | 0.05 | 0.05 | 0.05 |
| Vitamin E | — | — | — | — | 0.01 | 0.01 | 0.01 | 0.01 | — |
| Octyl methoxycinnamate | — | — | — | — | — | — | 0.09 | 0.09 | — |
| Benzophenone-3 | — | — | — | — | — | — | 0.09 | 0.09 | — |

| | |
|---|---|
| Deionized Water | q.s. to 100% |

Definitions of Components
*1 High molecular weight water-soluble cationic polymer-1: Poly(2-acryloyloxyethyltrimethyl ammonium chloride-co-acrylamide) having a molecular weight of 10,000,000 and having a molar percentage of 2-acryloyloxyethyltrimethyl ammonium chloride monomer of 90-99%.
*2 High molecular weight water-soluble cationic polymer-2: Poly(2-acryloyloxyethyltrimethyl ammonium chloride-co-acrylamide) having a molecular weight of 15,000,000 and having a molar percentage of 2-acryloyloxyethyltrimethyl ammonium chloride monomer of 90-99%
*3 High molecular weight water-soluble cationic polymer-3: Poly(2-acryloyloxyethyltrimethyl ammonium chloride-co-acrylamide) having a molecular weight of 15,000,000 and having a molar percentage of 2-acryloyloxyethyltrimethyl ammonium chloride monomer of 70-90%
*4 High molecular weight water-soluble cationic polymer-4: Poly(2-acryloyloxyethyltrimethyl ammonium chloride-co-acrylamide) having a molecular weight of 15,000,000 and having a molar percentage of 2-acryloyloxyethyltrimethyl ammonium chloride monomer of 50-70%
*5 High molecular weight water-soluble cationic polymer-5: Poly(2-acryloyloxyethyltrimethyl ammonium chloride-co-acrylamide) having a molecular weight of 20,000,000 and having a molar percentage of 2-acryloyloxyethyltrimethyl ammonium chloride monomer of 90-99%
*6 High molecular weight water-soluble cationic polymer-6: Poly(2-methacryloyloxyethyltrimethyl ammonium chloride-co-acrylamide) having a molecular weight of 15,000,000 and having a molar percentage of 2-acryloyloxyethyltrimethyl ammonium chloride monomer of 90-99%
*7 Dimethicone blend: a blend of dimethicone having a viscosity of 18,000,000 mPa · s and dimethicone having a viscosity of 200 mPa · s available from GE Toshiba
*8 Dimethicone/Cyclomethicone: a blend dimethicone having a viscosity of 18,000,000 mPa · s and cyclopentasiloxane available from GE Toshiba
*9 Aminosilicone-1: BX3083-1 available from GE Toshiba
*10 Kathon CG: Available from Rohm&Haas
*11 Aminosilicone-2: Terminal aminosilicone which is available from GE having a viscosity of about 10,000 mPa · s, and having following formula: (R₁)ₐG₃₋ₐ—Si—(—OSiG₂)ₙ—(—OSiG_{b}(R₁)_{2-b})ₘ—O—SiG₃₋ₐ(R₁)ₐ
wherein
G is methyl;
a is an integer of 1;
b is 0, 1 or 2, preferably 1;
n is a number from 400 to about 600;
m is an integer of 0;
R₁ is a monovalent radical conforming to the general formula CqH_{2q}L,
wherein
q is an integer of 3 and
L is —NH₂

Method of Preparation

The conditioning compositions of “Ex. 1” through “Ex. 17” as shown above can be prepared by any conventional method well known in the art. They are suitably made as follows:

Cationic surfactants and high melting point fatty compounds are added to water with agitation, and heated to about 80° C. The mixture is cooled down to about 55° C. The high molecular weight water-soluble cationic polymer can be added to the mixture with agitation at about 55° C., or prior to the cooling down (i.e. at about 80° C.). If included, silicone compounds, perfumes, preservatives are added to the mixture with agitation. Then the mixture is cooled down to room temperature.

Examples 1 through 17 are hair conditioning compositions of the present invention which are particularly useful for rinse-off use. The embodiments disclosed and represented by the previous “Ex. 1” through “Ex. 17” have many advantages. For example, they can provide another benefit while maintaining improved conditioning benefits of gel matrix such as slippery feel during the application to wet hair and softness and moisturized feel on dry hair, and/or they can provide improved conditioning benefits especially wet conditioning benefits while maintaining the above dry conditioning benefits. Especially, the compositions of Examples 15-17 provide improved clean rinse feel during and after rinsing the hair, so that consumers can easily leave the hair and/or hands with a clean rinse feel, while maintaining improved conditioning benefits of gel matrix such as slippery feel during the application to wet hair and softness and moisturized feel on dry hair.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A hair conditioning composition comprising by weight:
(a) from about 0.01% to about 10% of a high molecular weight water-soluble cationic polymer having a molecular weight of about 5,000,000 or more;
(b) a gel matrix comprising: from about 0.1% to about 10% by weight of the composition of a cationic surfactant where in the cationic surfactant is a mono-long alkyl quaternized ammonium; from about 4.5% to about 20% by weight of the composition of a high melting point fatty compound; and an aqueous carrier.

2. The hair conditioning composition of claim 1 wherein the high molecular weight water-soluble cationic polymer has a molecular weight of about 10,000,000 or more.

3. The hair conditioning composition of claim 1 wherein the high molecular weight water-soluble cationic polymer comprises a water-soluble cationic monomer.

4. The hair conditioning composition of claim 3 wherein the high molecular weight water-soluble cationic polymer has a molar percentage of the water-soluble cationic monomer of from about 20% to about 100%.

5. The hair conditioning composition of claim 1 wherein the high molecular weight water-soluble cationic polymer further comprises a water-soluble nonionic monomer.

6. The hair conditioning composition of claim 5 wherein the high molecular weight water-soluble cationic polymer is poly(2-acryloyloxyethyltrimethyl ammonium chloride-co-acrylamide).

7. The hair conditioning composition of claim 1 wherein the high molecular weight water-soluble cationic polymer is a non-crosslinked polymer.

8. The hair conditioning composition of claim 1 wherein the composition is substantially free of anionic surfactants and anionic polymers.

9. The conditioning composition of claim 1 further comprising from about 0.1% to about 20% of a silicone compound.
